(19) United States
(12) Patent Application Publication
(10) Pub. No.: US 2012/0107321 A1
(43) Pub. Date: May 3, 2012

(54) Antibodies And Epitopes Specific To Misfolded Prion Protein

(75) Inventor: Neil R. Cashman, Vancouver (CA)

(73) Assignee: THE UNIVERSITY OF BRITISH COLUMBIA, Vancouver, BC (CA)

(21) Appl. No.: 13/254,047

(22) PCT Filed: Mar. 2, 2010
(86) PCT No.: PCT/CA2010/000303

§ 371 Date: Nov. 21, 2011

Related U.S. Application Data
(60) Provisional application No. 61/156,807, filed on Mar. 2, 2009.

(30) Foreign Application Priority Data
Oct. 6, 2009 (CA) PCT CA2009 001413

(51) Int. Cl. A61K 39/395; C07K 5/087; C07K 7/06; C07K 5/103; C07K 5/107; C07K 17/02; A61K 39/00; C12N 5/16; G01N 33/566; C12P 21/08; G01N 33/574; A61P 25/00; A61P 35/00; A61P 25/20; A61P 43/00; A61P 25/28; A61P 37/04; C07K 16/18

(52) U.S. Cl. 424/139.1; 530/387.9; 530/391.3; 530/391.7; 530/331; 530/329; 530/330; 424/178.1; 424/185.1; 435/331; 435/7.92; 435/69.6; 435/7.23

(57) ABSTRACT

The present invention relates to antibodies and immunogenic peptides specific to misfolded prion protein (PrP, e g, PrP^{Sc}), and uses thereof. The immunogenic peptides comprise the amino acid sequence tyrosine-methionine-leucine (YML). The antibodies or peptides can be used for treating or preventing a disease or disorder associated with misfolded PrP, including cancer. In particular, a IgM monoclonal antibody designated “1A1” was generated using a peptide consisting of the sequence GGYMLGS (i e, SEQ ID NO 8), which corresponds to residues 126-132 of human PrP 1A1 recognizes misfolded PrP, but not normal PrP.

### FIELD OF INVENTION

The present invention relates to antibodies and epitopes specific to misfolded prion protein. More specifically, the invention provides antibodies and epitopes specific to a YML epitope of a misfolded prion protein.

### BACKGROUND OF THE INVENTION

The prion diseases (e.g., Creutzfeldt-Jakob disease, bovine spongiform encephalopathy, sheep scrapie, and chronic wasting disease of deer and elk) are generally characterized by the template-directed conversion of normal cellular prion protein (PrP^{C}) into an abnormal, protease-resistant isoform (PrP^{Sc}). Some prion disease may be inherited, and may comprise a mutation in the PNRP gene, while others are sporadic or infectious. A variety of mutations have been identified in the heritable forms, and the mutations may render the PrP^{C} more susceptible to change to the abnormal and disease-associated PrP^{Sc} form.

The translation product of the PNRP gene generally consists of 253 amino acids in humans, 254 in hamster and mice or 256 amino acids in sheep and may undergo several post-translational modifications (e.g., Pucket, C. et al., Am. J. Hum. 49:320-329 (1991)). For example, in hamsters, a signal peptide of 22 amino acids is cleaved at the N-terminus, 23 amino acids are removed from the C-terminus on addition of a glycosyl phosphatidylinositol (GPI) anchor, and asparagine-linked oligosaccharides are attached to residues 181 and 197 in a loop formed by a disulfide bond (e.g., Stahl, N. et al., Biochemistry 29:5405-5412 (1990); Safar, J. et al., Proc. Natl. Acad. Sci. USA 87:6377, (1990)). In prion-related encephalopathies, PrP^{C} (normal cellular isoform) is converted into an altered form designated PrP^{Sc}, that can be experimentally distinguished from PrP^{C} based on, for example, one or more of the following characteristics: (1) PrP^{Sc} is insoluble in physiological solvents and forms aggregates; (2) PrP^{Sc} is partially resistant to proteolytic degradation by proteinase K in that only the N-terminal ˜67 amino acids are removed by proteinase K digestion under conditions in which PrP^{C} is completely degraded, and which results in a N-terminally truncated form known as PrP27-30; (3) PrP^{Sc} has an alteration in protein conformation, from alpha-helical for PrP^{C} to an altered form which is rich in beta-sheet secondary structure (e.g., Cohen et al. Science 264:530-531 (1994).

That structure plays a role in the conversion of the PrP^{C} to the PrP^{Sc} isoforms is well known, however specifics of the structure of the PrP^{Sc} isoform have been slower in coming due in part to difficulties relating to solubilization and the disordered structure of PrP^{Sc} aggregates. In human PrP^{C}, structure elements include beta strand 1 (residues 128-131), alpha helix 1 (residues 144-154), beta strand 2 (residues 161-164), alpha helix 2 (residues 173-194), and alpha helix 3 (residues 200-228) (Riek et al., 1996, Nature 382:180; Zahn 2000, Proc. Natl. Acad. Sci. 97:145-150). Knaus et al., 2001 (Nature Structural Biology 8:770-774) added to this body of knowledge by describing a possible mechanism for oligomerization in prion proteins via interaction and rearrangement of some structural elements.

As the PrP^{C} and PrP^{Sc} isoforms share the same amino acid sequence, stimulating an immune response in a healthy individual, or providing a therapeutic agent that interacts with both isoforms may at the least be ineffective, and may possibly be deleterious to the subject. It has been reported that the normal cellular isoform of the prion protein (PrP^{C}) is poorly immunogenic. Further, it has been reported that while antibodies that are preferentially reactive against PrP^{C} can interfere with prion propagation in vitro and in vivo, immune recognition of this essentially ubiquitous cell surface protein could be deleterious.

Conversion of prion protein in disease is associated with the loss of certain molecular surface epitopes, and the acquisition of others. Paramithiotis et al. (Nat Med 2003 9:893-899) describe a tripeptide motif YYR. U.S. Pat. No. 7,041,807 describes antibodies to a YYR epitope of a mammalian prion protein, and discusses YYX epitopes. U.S. Pat. No. 6,765,088 describes antibodies to fragments of bovine PrP. U.S. Pat. No. 5,846,533 describes antibodies specific for native PrP^{Sc} proteins, that are produced by a phage display methodology.

### SUMMARY OF THE INVENTION

The invention provides, in part, antibodies and epitopes specific to misfolded prion protein, for example, antibodies and epitopes specific to a YML epitope of a misfolded prion protein.

In one aspect, the invention provides an antibody or fragment thereof that binds a YML epitope of a misfolded PrP.

In an alternative embodiment, the antibody selectively binds a PrP^{Sc}.

In an alternative embodiment, the antibody does not specifically bind a PrP^{C}.

In an alternative embodiment, the epitope is present in a sequence selected from one or more of the group consisting of: GGYMLGS, GGYMLG, GYMLGS, GGYML, YMLGS, GYML and YMLG (SEQ ID NOs: 8-14).

In an alternative embodiment, the antibody is a monoclonal antibody.

In an alternative embodiment, the antibody is a polyclonal antibody.

In an alternative embodiment, the antibody is an IgG, IgM, IgE, IgD, or IgA.

In an alternative embodiment, the antibody may be produced by culturing the hybridoma deposited with the International Depositary Authority of Canada under accession number 260210-01.

In another aspect, the invention provides an immunoconjugate comprising an antibody or fragment thereof that binds a YML epitope of a misfolded PrP, and, conjugated therewith, an agent selected from one or more of a detectable label and a cytotoxin.

In another aspect, the invention provides an immunogenic peptide directed against an antibody that binds selectively to misfolded PrP, the peptide comprising a YML sequence.

In an alternative embodiment, the peptide may be useful to raise an antibody that binds selectively to a misfolded PrP selected from one or more of the group consisting of the sequence of SEQ ID NO: 7, SEQ ID NO:8, SEQ ID NO:9, SEQ ID NO:10, SEQ ID NO: 11, SEQ ID NO: 12, SEQ ID NO:13 or SEQ ID NO:14.

In an alternative embodiment, the peptide is not a full-length PrP protein.

In an alternative embodiment, the peptide may further comprise an immunogenic carrier to enhance immunogenicity of said peptide.

In another aspect, the invention provides a composition comprising an antibody or fragment thereof that binds a YML epitope of a misfolded PrP.

In another aspect, the invention provides a composition comprising an immunoconjugate comprising an antibody or fragment thereof that binds a YML epitope of a misfolded PrP, and, conjugated therewith, an agent selected from one or more of a detectable label and a cytotoxin.

In another aspect, the invention provides a composition comprising a peptide directed against an antibody that binds selectively to misfolded PrP, the peptide comprising a YML sequence. In an alternative embodiment, the peptide may be selected from one or more of the group consisting of the sequence of SEQ ID NO: 7, SEQ ID NO:8, SEQ ID NO:9, SEQ ID NO:10, SEQ ID NO: 11, SEQ ID NO: 12, SEQ ID NO:13 or SEQ ID NO:14.

In an alternative embodiment, the peptide may further comprise an immunogenic carrier to enhance immunogenicity of said peptide.

In an alternative embodiment, the composition may be a pharmaceutical composition.

In an alternative embodiment, the composition may further comprise a pharmaceutical carrier.

In another aspect, the invention provides a use of the antibody or fragment thereof, the immunoconjugate, the peptide, or the composition, for the treatment of a disease or disorder associated with misfolded PrP.

In another aspect, the invention provides a use of a vaccine comprising the peptide or the immunoconjugate, for the treatment of a disease or disorder associated with misfolded PrP.

In another aspect, the invention provides a use of the antibody or fragment thereof, the immunoconjugate, the peptide, or the composition, for the treatment of a disease or disorder associated with PrP^{Sc}.

In another aspect, the invention provides a use of a vaccine comprising the peptide or the immunoconjugate, for the treatment of a disease or disorder associated with PrP^{Sc}.

In an alternative embodiment, the disease or disorder may be selected from Gerstmann-Sträussler-Scheinker disease (GSS), familial Creutzfeldt-Jakob disease, sporadic Creutzfeldt-Jakob disease, iatrogenic Creutzfeldt-Jakob disease, variant Creutzfeldt-Jakob disease, fatal familial insomnia, scrapie, Kuru, spongiform encephalopathy, transmissible mink encephalopathy, chronic wasting disease, feline spongiform encephalopathy, and exotic ungulate encephalopathy.

In another aspect, the invention provides a use of the antibody or fragment thereof, the immunoconjugate, the peptide, or the composition, for the treatment of a tumour comprising a tumorigenic cell expressing a misfolded PrP.

In another aspect, the invention provides a use of a vaccine comprising the peptide or the immunoconjugate, for the treatment of a tumour comprising a tumorigenic cell expressing a misfolded PrP.

In an alternative embodiment, the tumour may have a YML+ phenotype.

In another aspect, the invention provides a method of treating or preventing a disease or disorder associated with misfolded PrP, the method comprising administering a therapeutically effective amount of a the antibody or fragment thereof, the immunoconjugate, the peptide, or the composition, to a subject in need thereof.

In another aspect, the invention provides a method of immunizing a subject with, or at risk for, a disease or disorder associated with misfolded PrP, the method comprising administering a therapeutically effective amount of a vaccine comprising the peptide, to a subject in need thereof.

In an alternative embodiment, the disease or disorder is associated with PrP^{Sc}.

In an alternative embodiment, the disease or disorder is selected from Gerstmann-Sträussler-Scheinker disease (GSS), familial Creutzfeldt-Jakob disease, sporadic Creutzfeldt-Jakob disease, iatrogenic Creutzfeldt-Jakob disease, variant Creutzfeldt-Jakob disease, fatal familial insomnia, scrapie, Kuru, spongiform encephalopathy, transmissible mink encephalopathy, chronic wasting disease, feline spongiform encephalopathy, and exotic ungulate encephalopathy.

In another aspect, the invention provides a method for the treatment of a tumour comprising a tumorigenic cell expressing a misfolded PrP, the method comprising administering a therapeutically effective amount of a the antibody or fragment thereof, the immunoconjugate, the peptide, or the composition, to a subject in need thereof.

In an alternative embodiment, the tumor may have a YML+ phenotype.

In another aspect, the invention provides a hybridoma cell line that produces a monoclonal antibody that binds to a YML epitope of a misfolded PrP.

In an alternative embodiment, the misfolded PrP is a PrP^{Sc}.

In an alternative embodiment, the hybridoma cell line is the hybridoma deposited with the International Depositary Authority of Canada under accession number 260210-01, and progeny and derivatives thereof.

In an alternative embodiment, the YML epitope is present in sequence GGYMLGS, GGYMLG, GYMLGS, GGYML, YMLGS, GYML and YMLG (SEQ ID NOs: 8-14).

In another aspect, the invention provides a method for detecting a misfolded PrP in a biological sample, comprising: (a) contacting a biological sample with the antibody of or fragment thereof, or the immunoconjugate, under conditions that allow for the formation of a complex between said antibody or said immunoconjugate and said misfolded PrP, and (b) detecting the complex as an indication that misfolded PrP is present in the biological sample.

In an alternative embodiment, the complex is detected by immunoblotting.

In an alternative embodiment, the misfolded PrP is a PrP^{Sc}.

In another aspect, the invention provides a method of producing an antibody that binds a YML epitope of a misfolded PrP, the method comprising: (a) culturing a hybridoma cell line that produces a monoclonal antibody that binds to a YML epitope of a misfolded PrP under conditions that release the antibody into the culture supernatant; and (b) isolating the antibody from the supernatant.

In an alternative embodiment, the cultured hybridoma is the hybridoma having accession number 260210-01.

In another aspect, the invention provides a method of producing an antibody that binds a YML epitope of a misfolded PrP, the method comprising: (a) immunizing a subject with the peptide; and (b) isolating the antibody from a tissue of the subject, or from a hybridoma prepared from the tissue.

In another aspect, the invention provides a kit for detecting the presence of misfolded PrP in a biological sample comprising: (a) one or more antibodies or antisera that specifically bind the YML epitope of misfolded PrP; and (b) instructions for its use.

In an alternative embodiment, the kit may further comprise one or more detection reagents.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows the immunoprecipitation of mouse and hamster brain homogenates using magnetic bead-coupled PrP specific monoclonal antibodies and controls (including antibodies that recognize both isoforms of the prion protein, and other antibodies that recognize neither isoform of the prion protein), followed with detection using a monoclonal antibody (6D11 coupled to biotin) that recognizes both PrP^{C} and PrP^{Sc}. Hamster WT—normal hamster brain homogenate; RML—brain homogenate from RML mouse-adapted prion infected mice; Tg20—brain homogenate from PrP^{C} overexpressing mouse strain; K/O—brain homogenate from PrP^{C}−/− mouse strain; WT—brain homogenate from wild type (uninfected, normal) mouse; 263K—brain homogenate from 263K hamster-adapted prion infected hamster; control—PrP^{Sc} protein. 8B4 beads—brain homogenates immunoprecipitated with 8B4 antibody—coupled beads (recognizes both PrP^{C} and PrP^{Sc}); 1A1 beads—brain homogenates immunoprecipitated with 1A1 antibody coupled beads (recognizes PrP^{Sc} protein); 4E4 beads—brain homogenates immunoprecipitated with 4E4 antibody-coupled beads (recognizes unrelated protein); IgM isotype beads—brain homogenates immunoprecipitated with IgM isotype negative control antibody; only beads—brain homogenates immunoprecipitated with beads only (no antibody).

FIG. 2 shows amino acid sequences of A) human, B) sheep, C) mouse, D) hamster, E) bovine and F) elk prion protein (SEQ ID NOs: 1-6).

FIG. 3 shows a Clustal W alignment of the human, sheep, mouse, hamster and bovine sequences of FIG. 2.

FIG. 4 provides flow cytometry histograms of results with normal and tumour cells probed with either isotype control antibody (dark shading), or with PrP antibody 6D11 or YML-specific antibody 1A1 (black line, as indicated).

FIG. 5 shows the effect of treatment with 1A1 antibody on B16-F10 tumour bearing mice.

### DETAILED DESCRIPTION

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

A “prion” refers to an agent that is composed largely, and perhaps solely of a single protein, the “prion protein” or “PrP.” Misfolded prion protein (misfolded PrP) has been implicated in a variety of diseases. Normal cellular prion protein is generally referred to as PrP^{C}, while a misfolded protease-resistant isoform is referred to as PrP^{Sc}. PrPs have been identified in a number of species, including mammalian and avian species. Exemplary mammalian PrPs are described in SEQ ID NOs: 1-6.

The term “epitope” refers to an arrangement of amino acids in a protein or modifications thereon (for example glycosylation). The amino acids may be arranged in a linear fashion, such as a primary sequence of a protein, or may be a secondary or tertiary arrangement of amino acids in close proximity once a protein is partially or fully configured. Epitopes may be specifically bound by an antibody, antibody fragment, peptide, peptidomimetic or the like, or may be specifically bound by a ligand. An epitope may have a range of sizes—for example a linear epitope may be as small as two amino acids, or may be larger, from about 3 amino acids to about 20 amino acids. In some embodiments, an epitope may be from about 5 amino acids to about 10 or about 15 amino acids in length. An epitope of secondary or tertiary arrangements of amino acids may encompass as few as two amino acids, or may be larger, from about 3 amino acids to about 20 amino acids. In some embodiments, a secondary or tertiary epitope may be from about 5 amino acids to about 10 or about 15 amino acids in proximity to some or others within the epitope.

An “isoform” is any of several different forms of the same protein. The variant forms may result from one or more single nucleotide polymorphisms (e.g. resulting in a single amino acid change), or may be a result of splicing variants, for example including or excluding a sequence of amino acids in the translated protein. Variants may also result from differences in folding of the protein, so that one or more epitopes that are ‘buried’ within the 3 dimensional structure in one isoform, are exposed in a second isoform of the protein. These folding variants may be due to sequence differences, post translational modifications, or other influences, such as the presence of a particular isoform. The prion protein is an example of a protein with the same amino acid sequence being presented in two structural isoforms: PrP^{C} (the ‘normal’, ‘unaffected’, ‘native’ or ‘wild-type’ isoform) and the PrP^{Sc} isoform (the ‘disease-state’, ‘affected’, ‘misfolded’ or ‘abnormal’ isoform).

Exposure of misfolding-specific epitopes of the prion protein provides for one or more prion-specific epitopes that allow for differentiation between the PrP^{C} and misfolded isoforms, e.g., PrP^{Sc} isoforms, of the prion protein. These epitopes may be used as a diagnostic target (e.g. for use with ELISA or flow cytometry—based diagnostic methods to be performed on a biological sample from a subject having, or suspected of having a prion-associated disease or disorder. These epitopes may also be used to as a therapeutic or prophylactic target. For example, one or more of the epitopes may be used in a pharmaceutical composition for inducing immunity in a subject to whom it is administered, to prevent the propagation of prion misfolding that is found with prion-associated diseases or disorders. As another example, the one or more epitopes may be specifically bound by an immune molecule, such as an antibody, the immune molecule having been modified to convey a therapeutic agent to a cell or tissue comprising the misfolded prion protein.

Pruisner 1993 (Dev. Biol Stand. 80:31-44) provides a review of some prion diseases and disorders (alternately referred to as transmissible spongiform encephalopathies; TSE) of animals and humans. Diseases or disorders found in human or animals associated with prion protein misfolding (“prion-associated”, or “prion-misfolding associated”) include, but are not limited to, Gerstmann-Sträussler-Scheinker disease (GSS), familial Creutzfeldt-Jakob disease, sporadic Creutzfeldt-Jakob disease, iatrogenic Creutzfeldt-Jakob disease, variant Creutzfeldt-Jakob disease, fatal familial insomnia, scrapie (e.g. in sheep or goats), Kuru, bovine spongiform encephalopathy (mad cow disease), transmissible mink encephalopathy, chronic wasting disease (e.g. in deer, elk and moose), feline spongiform encephalopathy, exotic ungulate encephalopathy (e.g. in nyala, oryx, greater kudu), spongiform encephalopathy of the ostrich. The diseases or disorders associated with prion protein misfolding further include cancer, particularly cancers associated with cell types that have a PrP+ phenotype, which can ultimately present with surface epitopes associated uniquely with misfolded PrP, such as the YML epitope.

Two beta strands are present in the globular domain of PrP. The beta 1 strand (residues 128-131 using human sequence numbering) comprises a YML (SEQ ID NO:7) sequence. In the native PrP^{C} isoform (natively structured PrP^{C}), beta strand 1 is buried within the three-dimensional structure of the PrP^{C} isoform, and is not solvent-accessible for interaction with immune cells, antibodies or other molecules. Without being bound to any particular hypothesis, upon induction of the conformational shift that results in the misfolded form (e.g. by low-pH treatment, by exposure to PrP^{Sc} isoform, or other known methods of inducing the PrP^{C} to PrP^{Sc} rearrangement), the beta strand 1 may be exposed to the solvent and available for interaction with immune cells, antibodies or other molecules.

Amino acid sequences comprising the YML sequence and some or all of the amino acids comprising beta strand 1 of a mammalian PrP amino acid sequence and in some embodiments further comprising additional amino acids flanking beta strand 1, including but not limited to GGYMLGS (SEQ ID NO: 8), GGYMLG (SEQ ID NO: 9), GYMLGS (SEQ ID NO: 10), GGYML (SEQ ID NO: 11), YMLGS (SEQ ID NO: 12), GYML (SEQ ID NO: 13), YMLG (SEQ ID NO: 14) and YML (SEQ ID NO: 7).

Therefore, the invention provides for peptides comprising one or more than one of amino acid sequences SEQ ID NO: 7-14. More generally, the peptides useful herein are those that comprise and present the YML sequence as an epitope useful to raise antibodies that bind selectively to the YML. Such peptides can include the full length PrP protein but in a form that, necessarily, is misfolded so that the YML epitope is presented to the antibody production host. In practice, the YML-containing peptides will usually consist of not more than about 50 amino acid residues, e.g., not more than about 40 residues, 30 residues, 20 residues or 15 residues, where the choice of maximum residue number is made based on the desire to present the YML epitope in an immunogenic form while minimizing the cost associated with its production. The peptide will comprise a minimum number of residues, in addition to the YML sequence, sufficient to present YML as an immunogenic epitope against which antibodies can be raised. For instance, the YML-containing peptide will typically require at least about 5 residues, 6 residues or 7 residues. As noted herein, the peptide can be coupled to any agent useful to enhance its immunogenicity in the antibody production host.

Immunogenic peptides that include the YML epitope, such as peptides comprising one or more than one of SEQ ID NO: 7-14 may be used inducing an immune response in a subject, the immune response being specific to misfolded PrP, such as the PrP^{Sc} isoform. For example, such peptides may be used to immunize a mouse or another animal for the production of polyclonal (antisera) or monoclonal antibodies specific to misfolded PrP, such as the PrP^{Sc} isoform. Such antibodies may be used to detect misfolded PrP, such as PrP^{Sc} in a biological sample, for example, in immunological assays. Such peptides may be provided in a pharmaceutical preparation.

Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

A protein or polypeptide, or fragment or portion of a protein or polypeptide is specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol. Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity. For proteins with multiple isoforms, an isoform may be specifically identified when it is differentiated from other isoforms from the same or a different species, by specific detection of a structure, sequence or motif that is present on one isoform and is absent, or not detectable on one or more other isoforms.

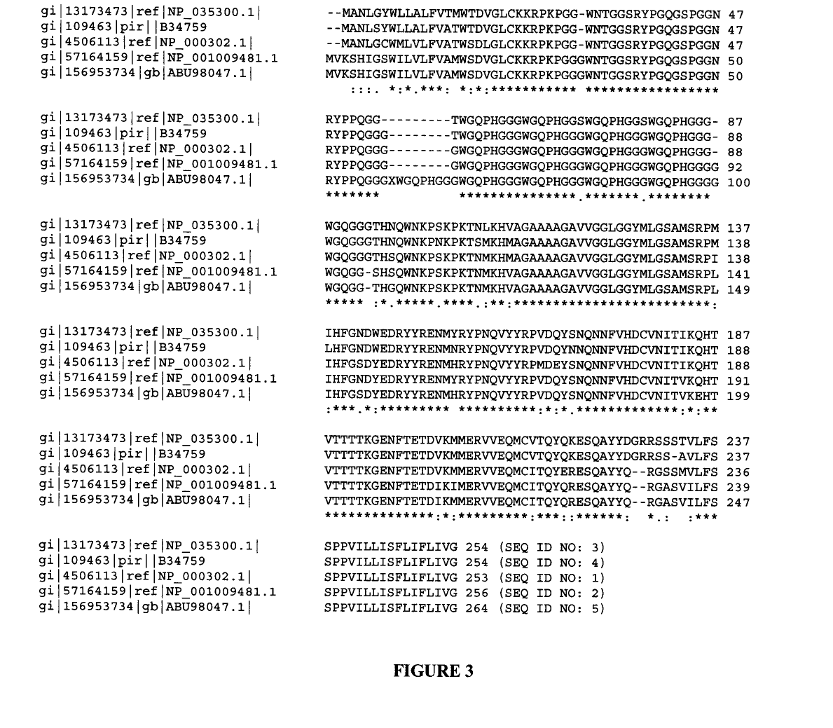

It will be appreciated by a person of skill in the art that any numerical designations of amino acids within a sequence are relative to the specific sequence. Also, the same positions may be assigned different numerical designations depending on the way in which the sequence is numbered and the sequence chosen. Furthermore, sequence variations such as insertions or deletions, may change the relative position and subsequently the numerical designations of particular amino acids at and around a site or element of secondary or tertiary structure. For example, the sequences represented by SEQ ID NOs: 1-6 all represent amino acid sequences of mammalian prion proteins from human, mouse, sheep, cow, hamster or elk. However, as is illustrated in FIG. 3, there may be some sequence differences, numbering differences between them, or sequence and numbering differences between them. It will also be apparent to one of skill in the art that the relative location of the epitopes, sequences and structural elements of the prion protein is the same in the various species. Other sequences representing prion protein sequences, wild-type or normal, or with or without mutations associated with some prion-misfolding associated diseases or disorders, may be identified by sequencing nucleic acid samples or protein samples (for example, using standard methods such as those referenced herein), or using any of the sequences listed herein, or a fragment of any of these in a BLAST search of a sequence database comprising one or more prion amino acid or nucleic acid sequences (mutant or normal, full, partial or fragments thereof). BLAST may also be used to identify prion protein sequences, or prion protein-like sequences in other species.

Nomenclature used to describe the peptide compounds of the present invention follows the conventional practice where the amino group is presented to the left and the carboxy group to the right of each amino acid residue. In the sequences representing selected specific embodiments of the present invention, the amino- and carboxy-terminal groups, although not specifically shown, will be understood to be in the form they would assume at physiologic pH values, unless otherwise specified. Each amino acid residue may be generally represented by a one-letter or three-letter designation, corresponding to the trivial name of the amino acid, in accordance with the following Table 1:

TABLE 1
Nomenclature and abbreviations of the 20 standard
L-amino acids commonly found in peptides:

| | Three -letter | One-letter |
|---|---|---|
| Full name | abbreviation | abbreviation |
| Alanine | Ala | A |
| Cysteine | Cys | C |
| Aspartic acid | Asp | D |
| Glutamic acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asp | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |

Standard reference works setting forth the general principles of immunology known to those of skill in the art include, for example: Harlow and Lane, Antibodies: A Laboratory Manual, 2d Ed., Cold Spring Harbor Laboratory Press, Cold Spring Harbor, N.Y. (1999); HARLOW and LANE, Using Antibodies: A Laboratory Manual. Cold Spring Harbor Laboratory Press, New York; COLIGAN et al. eds. Current Protocols in Immunology, John Wiley & Sons, New York, N.Y. (1992-2006); and Roitt et al., Immunology, 3d Ed., Mosby-Year Book Europe Limited, London (1993).

Standard reference works setting forth the general principles of recombinant DNA technology known to those of skill in the art include, for example: Ausubel et al, Current Protocols In Molecular Biology, John Wiley & Sons, New York (1998 and Supplements to 2001); Sambrook et al, Molecular Cloning: A Laboratory Manual, 2d Ed., Cold Spring Harbor Laboratory Press, Plainview, N.Y. (1989); Kaufman et al, Eds., Handbook Of Molecular And Cellular Methods In Biology And Medicine, CRC Press, Boca Raton (1995); McPherson, Ed., Directed Mutagenesis: A Practical Approach, IRL Press, Oxford (1991).

An “antibody”, as used herein, includes polyclonal antibodies from any native source, and native or recombinant monoclonal antibodies of classes IgG, IgM, IgA, IgD, and IgE, hybrid derivatives, humanized or chimeric antibodies, and fragments of antibodies including Fab, Fab′, and F(ab′)2, and the products of a Fab or other immunoglobulin expression library. The antibody may be naturally-occurring, e.g., isolated and/or purified from an animal (e.g., mouse, rabbit, goat, horse, chicken, hamster, human, or the like). The antibody can be in monomeric or polymeric form. The antibody, or antigen binding portion thereof, can be modified to comprise a detectable label, such as, for instance, biotin, a radioisotope, a fluorophore (e.g., fluorescein isothiocyanate (FITC), phycoerythrin (PE)), an enzyme (e.g., alkaline phosphatase, horseradish peroxidase), or an element particles (e.g., gold particles).

Antibodies and fragments that bind “selectively” to misfolded PrP, via the YML epitope exposed by misfolding, will bind misfolded PrP with an affinity that is at least one order of magnitude greater (e.g., at least 2, 3, 4 or 5 orders of magnitude greater) than the affinity with which they bind natively structured PrP. For instance, the binding affinity of the YML antibody for PrP^{Sc} is preferably at least an order of magnitude greater than its binding affinity for the PrP^{C} protein. Relative binding affinities can be determined, and the YML antibody so selected, on the basis of assays and techniques that generally are well established in the art for this purpose.

A hybridoma method may be used to make monoclonal antibodies (KOHLER et al. (1975) Nature 256:495). Alternately, monoclonal antibodies may be made by recombinant DNA methods (for example U.S. Pat. No. 4,816,567). Monoclonal antibodies may also be isolated from a phage antibody library, for example, by using the techniques described in CLACKSON et al. (1991) Nature 352:624-628; and MARLTS et al. 1991 J. Mol. Biol. 222:581-597. Methods of making and characterizing chimeric or humanized antibodies are known in the art, and are described in, for example, Kashmiri et al., 2005. Methods 36:25-34; Gonzales et al., 2005. Tumor biology 26:31-43. It may be advantageous to employ a PrP^{0/0} mouse (a ‘knockout mouse”) in the production of hybridomas specific to misfolded PrP, e.g., specific to PrP^{Sc} (see, for example, the methods provided by U.S. Pat. No. 6,765,088).

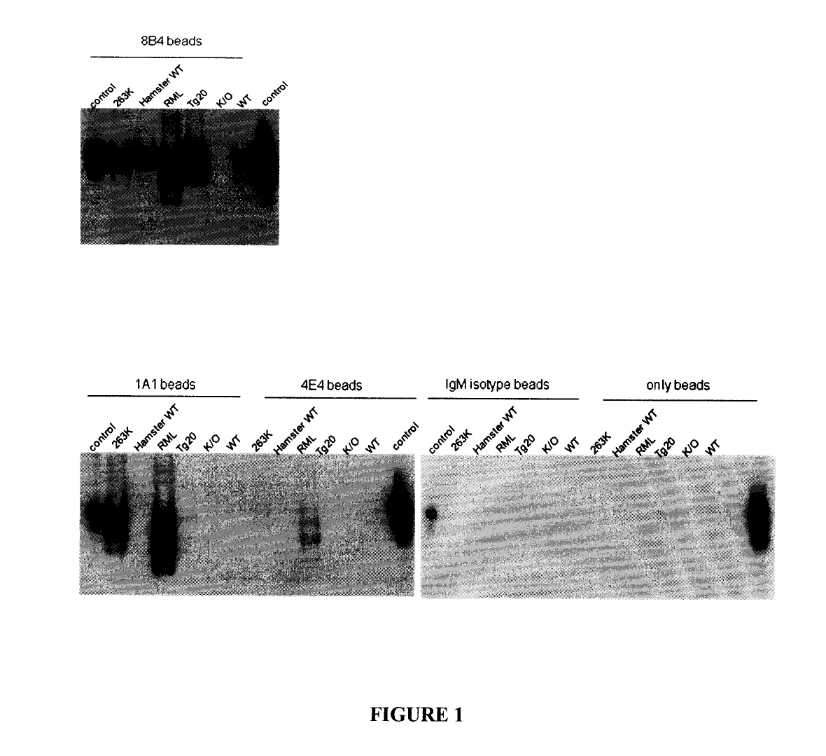

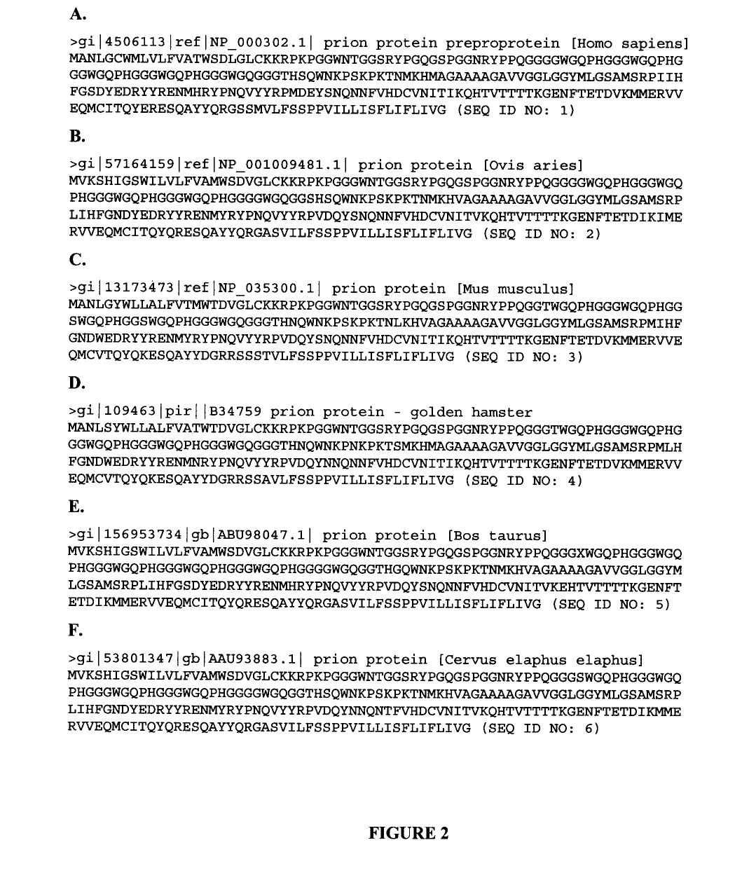

The inventor has generated a hybridoma and produced an IgM monoclonal antibody designated 1A1 using a peptide (Gly-Gly-Tyr-Met-Leu-Gly-Ser, SEQ ID NO: 8) comprising a sequence comprising amino acids found in beta strand 1. This sequence is conserved in prion-susceptible species including, but not limited to, human (SEQ ID NO: 1), mouse (SEQ ID NO: 3), bovine (SEQ ID NO: 5), hamster (SEQ ID NO: 4), ovine (SEQ ID NO: 2) and elk (SEQ ID NO: 6) (FIG. 3). The 1A1 monoclonal antibody specifically recognizes disease-misfolded isoform of the prion protein when compared with isotype control antibodies (FIG. 1).

Therefore, the invention provides for an antibody, or a fragment thereof, that binds an epitope comprising a YML sequence of a mammalian PrP amino acid sequence.

The invention further provides for an antibody, or a fragment thereof, that binds an epitope comprising a YML sequence of a mammalian PrP amino acid sequence, and wherein the antibody does not specifically bind natively structured PrP^{C}.

The invention further provides for an antibody that specifically binds an epitope found in whole or in part, on the beta strand 1 of mammalian PrP amino acid sequence.

The invention further provides for a hybridoma cell line that produces a monoclonal antibody that binds a YML epitope of a mammalian PrP amino acid sequence.

In a specific embodiment, the invention provides the hybridoma deposited under terms of the Budapest Treaty with the International Depositary Authority of Canada on Feb. 26, 2010, under accession number 260210-01, and all progeny and derivatives thereof, including derivatives that incorporate genes encoding the heavy and light chains, or sequences encoding the complementarity determining regions, of the antibody produced by the deposited hybridoma.

In another specific embodiment, the present invention provides the monoclonal antibody designated 1A1, obtained as a product of culturing the hybridoma referenced above. Also provided are YML-binding fragments of the 1A1 antibody. In related embodiments, the invention provides and embraces antibodies and their fragments that compete with the 1A1 antibody for binding to the YML epitope.

Antibodies according to various embodiments of the present invention may be used in assays or tests to determine the presence, absence or relative amount of PrP^{Sc} isoform in a biological sample. The biological sample may be obtained from a subject. Similarly, the antibodies can be used in assays or tests to determine the presence, absence or relative amount of tumour cells that present a misfolded form of PrP on their membrane surfaces.

Proteins or protein complexes may be specifically identified and quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, some chromatographic techniques (i.e. immunoaffinity chromatography), flow cytometry, immunoprecipitation and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, sequencing, protease digests, and the like. Such methods are based on the mass, charge, hydrophobicity or hydrophilicity, which is derived from the amino acid complement of the protein or protein complex, and the specific sequence of the amino acids. Immunologic and non-immunologic methods may be combined to identify or characterize a protein or protein complex.

Standard reference works described herein and known to those skilled in the relevant art describe both immunologic and non-immunologic techniques, their suitability for particular sample types, antibodies, proteins or analyses.

In some embodiments of the invention, a tissue extract, or homogenate comprising PrP^{Sc} or cell-associated forms of misfolded PrP^{C}, may be combined with and allowed to interact with an antibody that binds to YML-presenting forms of PrP, such as PrP^{Sc}, under conditions that allow for complex formation between the antibody and prion protein. The antibody may be bound to a support matrix, for example a plastic or magnetic bead. The bound protein complex is collected (e.g. by centrifugation or by magnetic collection), washed, denatured and subjected to gel electrophoresis. Following gel electrophoresis, the proteins are subject to western blotting and the blot probed with various antibodies that may include one or more controls, one or more antibody specific for the PrP^{C} isoform and one or more antibody specific for the PrP^{Sc} isoform. If PrP^{Sc} isoform is present in the sample, the PrP^{Sc} specific antibody will identify the presence of the PrP^{Sc} isoform in the sample. If PrP^{C} isoform is also present in the sample, an antibody that detects only the PrP^{C} isoform will identify the presence of the PrP^{C} isoform. In some embodiments, the detection of the PrP^{Sc} and PrP^{C} isoforms is quantitative, or semi-quantitative, thus it may be possible to obtain an estimate of the relative ratio of PrP^{C} and PrP^{Sc} in the sample.

In embodiments, the biological sample investigated for YML immunoreactivity is a blood sample or YML antibody-reactive fraction thereof, in the case where detection is aimed at diagnosis either of PrP^{Sc}-related neurologic disease or blood-borne cancer. In the alternative, the biological sample is a tissue sample or homogenate thereof, such as a tumour biopsy, in the case where detection is aimed at diagnosis of solid cancers.

Therefore, the invention provides for a method of detecting PrP^{Sc} in a biological sample, the method comprising contacting a biological sample with an antibody that binds PrP^{C} and PrP^{Sc} under conditions that allow for complex formation, and detecting the presence of a PrP^{Sc} isoform in the complex. Similarly, the invention provides a method for detecting any misfolded form of PrP that is immunoreactive with a YML antibody, in a biological sample.

The term “subject” or “patient” generally refers to mammals and other animals including humans and other primates, companion animals, zoo, and farm animals, including, but not limited to, cats, dogs, rodents, rats, mice, hamsters, rabbits, horses, cows, sheep, pigs, elk or other ungulates, goats, poultry, etc. A subject includes one who is to be tested, or has been tested for prediction, assessment or diagnosis of a disease or disorder associated with prion protein misfolding. The subject may have been previously assessed or diagnosed using other methods, such as those described herein or those in current clinical practice, or may be selected as part of a general population (a control subject). A subject may be a transgenic animal, e.g. a rodent, such as a mouse, that comprises a PrP^{C} or PrP^{Sc} isoform, or is lacking expression of a prion protein (e.g. a ‘knock-out’ mouse). For example, the subject may a transgenic mouse overexpressing a normal isoform (PrP^{C}) or may be a wild-type mouse or hamster that has been infected with a disease-associated isoform (PrP^{Sc}).

A “biological sample” or a “sample” refers generally to body fluid or tissue or organ sample from a subject. For example, the biological sample may a body fluid such as cerebrospinal fluid, blood, plasma, lymph fluid, serum, urine or saliva. A tissue or organ sample, such as that obtained from a solid or semi-solid tissue or organ, may be digested, extracted or otherwise rendered to a liquid form—examples of such tissues or organs include cultured cells, blood cells, brain, neurological tissue, skin, liver, heart, kidney, pancreas, islets of Langerhans, bone marrow, blood, blood vessels, heart valve, lung, intestine, bowel, spleen, bladder, penis, face, hand, bone, muscle, fat, cornea or the like, including tumourigenic forms thereof. A plurality of biological samples may be collected at any one time. A biological sample or samples may be taken from a subject at any time, including before the subject is diagnosed with, or suspected of having a prion-misfolding associated disease or disorder, during a therapeutic regimen for the treatment or amelioration of symptoms of a prion-misfolding associated disease or disorder, after death of the subject (regardless of the cause, or suspected cause). Alternately, a biological sample may include donated body fluid or tissue, such as blood, plasma or platelets when in care of a centralized blood supply organization or institution. Alternately, a biological sample may include meat, blood or tissue from a food animal, for example taken at the time of slaughter in an abattoir.

A sample may also include, without limitation, PrP^{C} or PrP^{Sc} protein isoforms produced in cell culture by normal or modified cells (e.g., via recombinant DNA technology). A sample may also be a cell or cell line created under experimental conditions, that are not directly isolated from a subject. A sample can also be cell-free, artificially derived or synthesized. A “control” includes a sample or standard obtained for use in determining the baseline e.g., expression or activity or occurrence. Accordingly, a control may be obtained from normal cells or tissue e.g., from a subject not affected by a prion-misfolding associated disease or disorder; from a subject not suspected of being at risk for prion-misfolding associated disease or disorder; or from cells or cell lines derived from such subjects, or extracts or homogenates thereof. A control may also be a standard, e.g., previously established standard. Accordingly, any test or assay conducted according to the invention may be compared with the standard; further it may not be necessary to obtain a control sample for comparison each time.

In another example, antibodies as described herein may be used in a pharmaceutical composition for the treatment, prophylaxis or amelioration of a prion-misfolding associated disease or disorder in a subject. A pharmaceutical composition comprising a therapeutically effective amount of an antibody according to some embodiments of the invention and a pharmaceutically acceptable excipient may be administered to a subject to treat a prion-misfolding associated disease or disorder. The antibody may inhibit the formation of PrP^{Sc} aggregates, inhibit intercellular communication or intracellular signaling via the misfolded PrP, or block the further conversion of PrP^{C} to PrP^{Sc} isoforms. The pharmaceutical composition may be useful, for example, in reducing a neurotoxic effect of PrP^{Sc} formation and/or aggregation. The pharmaceutical composition may further comprise an additive or agent that increases the permeability of the blood-brain barrier (for administration into the blood).

In another embodiment of the invention, an antibody may be used in the preparation of a medicament, for the treatment of a prion-misfolding associated disease or disorder. The antibody, or medicament or pharmaceutical composition comprising the antibody, may be used for the treatment of a prion-misfolding associated disease or disorder in a subject having, or suspected of having such a disease or disorder.

In another example, peptides comprising one or more than one of SEQ ID NO: 7-14 may be used in a pharmaceutical preparation for inducing an immune response in a subject, the immune response being specific to the PrP^{Sc} isoform. The pharmaceutical preparation may be useful as a vaccine.

In another embodiment of the invention, a peptide may be used in the preparation of a vaccine composition for the prevention or treatment of a prion-misfolding associated disease or disorder. The peptide, or medicament or vaccine composition comprising the peptide, may be used for the prevention or treatment of a prion-misfolding associated disease or disorder in a subject having, or suspected of having such a disease or disorder. The host-produced antibodies specific for a PrP^{Sc} isoform may prevent the aggregation of PrP^{Sc}, or may prevent the conversion of PrP^{C} to PrP^{Sc}.

The peptide may be coupled with a carrier to facilitate or enhance the host's immune response to the peptide. Examples of carriers are described, for example, in the standard references disclosed herein. The vaccine composition may further comprise one or more adjuvants, excipients or the like. Examples of adjuvants and excipients are described herein, and additional examples are described in, for example, the standard references described herein.

Standard reference works setting forth the general principles of medical physiology and pharmacology known to those of skill in the art include: Fauci et al., Eds., Harrison's Principles Of Internal Medicine, 14th Ed., McGraw-Hill Companies, Inc. (1998).

An “effective amount” of an antibody or peptide as used herein refers to (1) the amount of antibody in the pharmaceutical composition useful to reduce the effect of misfolded PrP in the recipient, such as to reduce the neurotoxic effect of PrP^{Sc} or to reduce the proliferative effect of tumours positive for misfolded PrP, and (2) the amount of peptide in a pharmaceutical composition to induce an immune response to a PrP^{Sc} isoform or misfolded PrP-presenting tumour cell in a subject. The effective amount may be calculated on a mass/mass basis (e.g. micrograms or milligrams per kilogram of subject), or may be calculated on a mass/volume basis (e.g. concentration, micrograms or milligrams per milliliter). Using a mass/volume unit, an antibody may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Quantities and/or concentrations may be calculated on a mass/mass basis (e.g. micrograms or milligrams per kilogram of subject), or may be calculated on a mass/volume basis (e.g. concentration, micrograms or milligrams per milliliter). Using a mass/volume unit, an antibody or peptide may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Compositions according to various embodiments of the invention, including therapeutic compositions, may be administered as a dose comprising an effective amount of an antibody or peptide. The dose may comprise from about 0.1 ug/kg to about 20 mg/kg (based on the mass of the subject), for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/kg, or any amount therebetween; or from about 1 ug/kg to about 2000 ug/kg or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000 ug/kg, or any amount therebetween; or from about 10 ug/kg to about 1000 ug/kg or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/kg, or any amount therebetween; or from about 30 ug/kg to about 1000 ug/kg or any amount therebetween, for example 30.0, 35.0, 40.0, 50.0 60.0, 70.0, 80.0, 90.0, 100, 120, 140, 160 180, 200, 250, 500, 750, 1000 ug/kg.

One of skill in the art will be readily able to interconvert the units as necessary, given the mass of the subject, the concentration of the pharmaceutical composition, individual components or combinations thereof, or volume of the pharmaceutical composition, individual components or combinations thereof, into a format suitable for the desired application.

The amount of a composition administered, where it is administered, the method of administration and the timeframe over which it is administered may all contribute to the observed effect. As an example, a composition may be administered systemically e.g. intravenous administration and have a toxic or undesirable effect, while the same composition administered subcutaneously may not yield the same undesirable effect. In some embodiments, localized stimulation of immune cells in the lymph nodes close to the site of subcutaneous injection may be advantageous, while a systemic immune stimulation may not.

Pharmaceutical compositions according to various embodiments of the invention may be formulated with any of a variety of physiologically or pharmaceutically acceptable excipients, frequently in an aqueous vehicle such as Water for Injection, Ringer's lactate, isotonic saline or the like. Such excipients may include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disentegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifiers, thickeners, preservatives, solubilizing agents, humectants, water, alcohols or the like. See, for example, Berge et al. (1977. J. Pharm Sci. 66:1-19), or Remington—The Science and Practice of Pharmacy, 21^{st} edition. Gennaro et al editors. Lippincott Williams & Wilkins Philadelphia (both of which are herein incorporated by reference).

Compositions comprising an antibody or peptide according to various embodiments of the invention may be administered by any of several routes, including, for example and without limitation, intrathecal administration, subcutaneous injection, intraperitoneal injection, intramuscular injection, intravenous injection, epidermal or transdermal administration, mucosal membrane administration, orally, nasally, rectally, topically or vaginally. Alternately, such compositions may be directly injected into a tumor, or a lymph node near a tumor, or into an organ or tissue near a tumor, or an organ or tissue comprising tumor cells. See, for example, Remington—The Science and Practice of Pharmacy, 21^{St} edition. Gennaro et al editors. Lippincott Williams & Wilkins Philadelphia. Carrier formulations may be selected or modified according to the route of administration.

Compositions according to various embodiments of the invention may be applied to epithelial surfaces. Some epithelial surfaces may comprise a mucosal membrane, for example buccal, gingival, nasal, tracheal, bronchial, gastrointestinal, rectal, urethral, vaginal, cervical, uterine and the like. Some epithelial surfaces may comprise keratinized cells, for example, skin, tongue, gingival, palate or the like.

Compositions according to various embodiments of the invention may be provided in a unit dosage form, or in a bulk form suitable for formulation or dilution at the point of use.

Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006. LL Brunton, editor. McGraw-Hill, New York, or Remington—The Science and Practice of Pharmacy, 21^{st} edition. Gennaro et al editors. Lippincott Williams & Wilkins Philadelphia.

Pharmaceutical compositions for use as vaccine compositions according to various embodiments of the invention may further comprise an adjuvant and administered as described. For example, a peptide for use in a vaccine composition may be combined with an adjuvant, examples of adjuvants include aluminum hydroxide, alum, Alhydrogel™ (aluminum trihydrate) or other aluminum-comprising salts, virosomes, nucleic acids comprising CpG motifs, squalene, oils, MF59, QS21, various saponins, virus-like particles, monophosphoryl-lipidA/trehalose dicorynomycolate, toll-like receptor agonists, copolymers such as polyoxypropylene and polyoxyethylene, or the like.

In the context of the present invention, the terms “treatment,”, “treating”, “therapeutic use,” or “treatment regimen” as used herein may be used interchangeably are meant to encompass prophylactic, palliative, and therapeutic modalities of administration of the compositions of the present invention, and include any and all uses of the presently claimed compounds that remedy a disease state, condition, symptom, sign, or disorder caused by an inflammation-based pathology, infectious disease, allergic response, hyperimmune response, or other disease or disorder to be treated, or which prevents, hinders, retards, or reverses the progression of symptoms, signs, conditions, or disorders associated therewith.

### Other Embodiments

In another embodiment, the invention provides for a method for identifying a compound for treatment of a prion-misfolding associated disease or disorder. An antibody specific for a YML epitope presented by a PrP^{Sc} isoform or other form of misfolded PrP is combined with a sample comprising the targeted antigen, e.g, PrP^{Sc}, as described herein, in the presence and absence of a test compound. The complex of the bound antibody and PrP^{Sc} is collected and analyzed as described herein for the relative amount of PrP^{Sc} in the complex. A level of binding of the PrP^{Sc} specific antibody in the presence of the test compound that is less than the level of binding in the absence of the test compound is indicative that the test compound may be a potential therapeutic compound for the treatment or amelioration of a prion-misfolding associated disease or disorder. In some embodiments, the antibody binds to an epitope comprising SEQ ID NO: 7.

In another embodiment, the invention provides for a method for clearing PrP^{Sc} from tissue or a composition intended for transplantation, oral consumption, or administering to a subject. For example, the composition or tissue is combined with one or more antibodies specific for mammalian PrP^{Sc} isoform, such that the PrP^{Sc} isoform is bound in a complex with the antibody. The bound isoform and antibody complex is subsequently separated from the tissue or composition, and the tissue or composition may be employed as intended.

### Articles of Manufacture

Also provided is an article of manufacture, comprising packaging material and a composition comprising an antibody or antisera specific for misfolded PrP such as mammalian PrP^{Sc}. The composition includes a physiologically or pharmaceutically acceptable excipient, and the packaging material may include a label which indicates the active ingredients of the composition (e.g. the antisera or antibody). The label may further include an intended use of the composition, for example as a diagnostic reagent to be used with kits as set out herein.

Also provided is an article of manufacture, comprising packaging material and a composition comprising a peptide according to one or more peptides as provided herein. The composition may include a physiologically or pharmaceutically acceptable excipient, and the packaging material may include a label which indicates the active ingredients of the composition (e.g. the peptide). The label may further include an intended use of the composition, for example as a therapeutic or prophylactic reagent, or as a composition to induce an immune response in a subject for the purpose of producing antisera or antibodies specific to mammalian PrP^{Sc}, to be used with kits as set out herein.

Kits

A kit comprising a composition comprising one or more peptides as provided herein, along with instructions for use of the compound or composition for the production or screening of antibodies for identification of a YML-specific antibody or antisera is provided. The kit may be useful for production and/or identification of YML specific antibodies or antisera, and the instructions may include, for example, dose concentrations, dose intervals, preferred administration methods, methods for immunological screening or testing, or the like.

In another embodiment, a kit for the preparation of a medicament, comprising a composition comprising one or more peptides as provided herein, along with instructions for its use is provided. The instructions may comprise a series of steps for the preparation of the medicament, the medicament being useful for inducing a therapeutic or prophylactic immune response in a subject to whom it is administered. The kit may further comprise instructions for use of the medicament in treatment for treatment, prevention or amelioration of one or more symptoms of a disease or disorder associated with prion misfolding. or in which prion misfolding is implicated, and include, for example, dose concentrations, dose intervals, preferred administration methods or the like.

In another embodiment, a kit for diagnosing a disease or disorder associated with prion misfolding is provided. The kit comprises one or more YML antibodies or antisera as described herein, along with instructions for its use. The antibody may further be coupled to a detection reagent. Examples of detection reagents include secondary antibodies, such as an anti-mouse antibody, an anti-rabbit antibody or the like. Such secondary antibodies may be coupled with an enzyme that, when provided with a suitable substrate, provides a detectable colorimetric or chemiluminescent reaction. The kit may further comprise reagents for performing the detection reaction, including enzymes such as proteinase K, blocking buffers, homogenization buffers, extraction buffers, dilution buffers or the like.

In another embodiment, a kit for detecting the presence of PrP^{Sc} in a biological sample is provided. The kit comprises one or more antibodies or antisera that specifically bind the PrP^{Sc} isoform of mammalian PrP as described herein, along with instructions for its use. The antibody may further be coupled to a detection reagent. Examples of detection reagents include secondary antibodies, such as an anti-mouse antibody, an anti-rabbit antibody or the like. Such secondary antibodies may be coupled with an enzyme that, when provided with a suitable substrate, provides a detectable colorimetric or chemiluminescent reaction. The kit may further comprise reagents for performing the detection reaction, including enzymes such as proteinase K, blocking buffers, homogenization buffers, extraction buffers, dilution buffers or the like.

The YML epitope is also useful as a target for cancer theranostics. As exemplified herein, certain tumour cell lines present an antigen that is reactive with antibodies raised against this epitope. These are cell lines that, while PrP+, clearly present a misfolded form of PrP recognized by YML antibody. Thus, the antibodies of the present invention are useful per se or as immunoconjugates for the detection and therapeutic targeting of YML+ tumour cells. The YML-containing vaccines similarly are useful for cancer treatment. The tumour cells including solid tumours and liquid tumours. As exemplified herein, the tumour targets are those that present the YML epitope, and therefore are those that present PrP on their surface, but in a misfolded form that reveals the YML epitope. Such tumors may be described as “YML+” or as having or comprising a YML+ phenotype. Tumours shown to present YML include those arising from lymphoid tissue, as represented by cell line MOLT-4, and from oligodendroglial lineages as represented by cell line MO3.13, as well as melanoma cells as represented by cell line B16. Of course, still other tumours that can be targeted with YML antibody can first be revealed using the YML antibody screens described above and in the examples herein.

The term “tumour cells” is used herein with reference to cancer cells, and tumours comprising such cells, that are characterized by unregulated cell growth. Tumour cells are thus characterized by neoplastic cell growth and proliferation, whether malignant or benign, and include all pre-cancerous and cancerous cells as well as tissues comprising such cells, including liquid and solid tumours. The term “tumour cells” includes human cancer cells and cancer cells from other mammals including pets, and livestock including horses, sheep, cattle and ungulates.

The present treatment method results in the inhibition of “growth or proliferation” of cancer cells presenting a YML epitope. At the in vitro level, inhibition of such growth or proliferation is revealed by a reduction in the number, size, viability, growth rate, proliferation rate, or metabolic activity of the cancer cells that are treated, relative to an untreated control sample. At the in vivo level, such inhibition of growth or proliferation can further be revealed as a reduction in the growth rate, size, number or metastatic status of tumours harbouring cancer cells that present the target epitope. It will be appreciated that all of these end-points can readily be determined using assays and procedures that are well established in the oncology field for this purpose, and with the aid of agents that detect the target epitope, as provided by the present invention and as detailed further herein.

To permit their use as cytotoxins per se, to inhibit directly the growth or proliferation of cancer cells presenting the YML epitope, the antibodies can exert their anti-cancer activity through endogenous mechanisms such as complement-mediated cytotoxicity (CDC) and/or antibody-dependent cellular cytotoxicity (ADCC). To this end, the YML antibody is optimally of the IgG1 isotype. It will be appreciated that the antibodies can be engineered or selected to have altered effector function, to enhance effectiveness in treating cancer. Cysteine residues, for instance, may be introduced to the Fc region to allow interchain disulfide bond formation. The resulting homodimeric antibody may have improved internalization capacity, and more importantly may have increased complement dependent cytotoxicity (CDC) and/or ADCC activities. Homodimeric antibodies with enhanced anti-tumour activity may also be prepared using heterobifunctional cross-linkers as described in Wolff et al, Cancer Research 53:2560-2565 (1993). Alternatively, an antibody can be engineered which has dual Fc regions and enhanced CDC and ADCC activity.

Antibody fragments useful in the present invention include the YML binding fragments of anti-YML antibodies, including Fab, Fab′, F(ab′)2, and Fv fragments, diabodies, linear antibodies, single chain antibody molecules, and multispecific antibodies formed from antibody fragments. Antibody fragments that incorporate the Fc region can also be engineered or conjugated as noted above to provide altered effector function, thereby to enhance ADCC and/or CDC activity. For cancer treatment, the YML antibodies and binding fragments thereof can also be provided and used as immunoconjugates, in which the antibody or fragment is coupled to a cytotoxin.

The immunoconjugates comprising the antibody may be conjugated to a variety of agents as noted above, including detectable labels including those useful in imaging, and drugs including cytotoxins. In embodiments, the conjugate comprises a cytotoxin and an agent that binds selectively to the YML epitope. “Cytotoxin” refers to a compound including a chemotherapeutic or a radiotherapeutic compound and the like that is useful therapeutically to reduce the viability of cancer cells, e.g., to inhibit the growth and/or proliferation of the cancer cells.

The YML antibody and the cytotoxin may be conjugated through non-covalent interaction, but more desirably, are coupled by covalent linkage either directly or, more preferably, through a suitable linker. In a preferred embodiment, the conjugate comprises a cytotoxin and a YML antibody, to form an immunoconjugate. Immunoconjugates of the antibody and cytotoxin are made using a variety of bifunctional protein coupling agents such as N-succinimidyl-3-(2-pyridyldithiol) propionate, iminothiolane, bifunctional derivatives of imidoesters such as dimethyl adipimidate HCL, active esters such as disuccinimidyl suberate, aldehydes such as glutaraldehyde, bis-azido compounds such as bis-(p-diazoniumbenzoyl)-ethylenediamine), diisocyanates such as tolyene 2,6-diisocyanate, and bis-active fluorine compounds (such as 1,5-difluoro-2,4-dinitrobenzene). Carbon-14-labeled 1-isothiocyanobenzyl-3-methyldiethylene triaminepentaacetic acid (MX-DTPA) is a chelating agent suitable for conjugation of radio nucleotide to the antibody.

The cytotoxin component of the immunoconjugate can be a chemotherapeutic agent, a toxin such as an enzymatically active toxin of bacterial, fungal, plant or animal origin, or fragments thereof, or a small molecule toxin), or a radioactive isotope such as ²¹²Bi, ¹³¹I, ¹¹¹In, ⁹⁰Y, and ¹⁸⁶Re, or any other agent useful to inhibit the growth or proliferation of a cancer cell.

Chemotherapeutic agents useful in the generation of such immunoconjugates include adriamycin, doxorubicin, epirubicin, 5-fluoroouracil, cytosine arabinoside (“Ara-C”), cyclophosphamide, thiotepa, busulfan, cytoxin, taxoids, e.g. paclitaxel, and docetaxel, toxotere, methotraxate, cisplatin, melphalan, vinblastine, bleomycin, etoposide, ifosgamide, mitomycin C, mitoxantrone, vincristine, vinorelbine, carboplatin, teniposide, daunomycin, caminomycin, aminopterin, dactinomycin, mitomycins, esperamicins, 5-FU, 6-thioguanine, 6-mercaptopurine, actinomycin D, VP-16, chlorambucil, melphalan, and other related nitrogen mustards. Also included are hormonal agents that act to regulate or inhibit hormone action on tumors such as tamoxifen and onapristone.

Toxins and fragments thereof which can be used include diphtheria A chain, non-bonding active fragments of diphtheria toxin, cholera toxin, botulinus toxin, exotoxin A chain (from Pseudomonas aeruginosa), ricin A chain, abrin A chain, modeccin A chain, alpha-sarcin, Aleurites fordii proteins, dianthin proteins, phytolaca Americana proteins (PAPI, PAPII, and PAP-S), momordica charantia inhibitor, curcin, crotin, sapaonaria, officinalis inhibitor, gelonin, saporin, mitogellin, restrictocin, phenomycin, enomycin, and the tricothcenes. Small molecule toxins include, for example, calicheamicins, maytansinoids, palytoxin and CC1065.

For treatment of subjects presenting with YML+ tumours, dosing can be carried out as noted hereinabove for treatment of PrP^{Sc} disorders. To identify subjects suitable for treatment, a biological sample comprising tumour cells can be screened also as described hereinabove to confirm the YML+ phenotype thereof. Alternatively, an image can be taken of the subject, either by localized or whole body imaging, after administration and accumulation of an immunoconjugate in which the YML antibody is coupled to an imaging agent, such as a technetium isotope (e.g., Tc⁹⁹), a gadolinium isotope, or the like.

Successful treatment of a subject presenting with a YML+ tumour is revealed as a reduction in YML+ tumour burden, such as a reduction in the number or distribution of YML+ tumours or in the size of a particular YML+ tumour, and/or by an enhancement of overall patient survival.

Other therapeutic regimens may be combined with the administration of the anti-cancer agents, e.g., vaccines, antibodies or conjugates, of the invention. For example, the patient to be treated with such anti-cancer agents may also receive radiation therapy, such as external beam radiation. Alternatively, or in addition, a chemotherapeutic agent may be administered to the patient. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration or the anti-tumor agent, e.g., antibody, or may be given simultaneously therewith. The antibody may be combined with an anti-estrogen compound such as tamoxifen or an anti-progesterone such as onapristone (see, EP 616812) in dosages known for such molecules.

It may be desirable to also administer antibodies or conjugates against other tumor associated antigens, such as antibodies which bind to the ErbB2, EGFR, ErbB3, ErbB4, or vascular endothelial factor (VEGF). Alternatively, or in addition, two or more antibodies binding that same or two or more different antigens disclosed herein may be co-administered to the patient. Sometimes it may be beneficial to also administer one or more cytokines to the patient. In a preferred embodiment, the antibodies herein are co-administered with a growth inhibitory agent. For example, the growth inhibitory agent may be administered first, followed by an antibody of the present invention. However, simultaneous administration or administration of the antibody of the present invention first is also contemplated. Suitable dosages for the growth inhibitory agent are those presently used and may be lowered due to combined action (synergy) of the growth inhibitory agent and the antibody herein. Further, the YML antibody or fragment may be administered in combination with a vaccine for raising YML antibody, providing both active and passive immunotherapy to the recipient.

A list of sequence identification numbers of the present invention is given in Table 2.

TABLE 2
List of Sequence Identification numbers.

| SEQ ID | | Figure reference |
|---|---|---|
| NO: | Description | (where relevant) |
| 1 | Human prion protein amino acid sequence | 2a |
| 2 | Sheep prion protein amino acid sequence | 2b |
| 3 | Mouse prion protein amino acid sequence | 2c |
| 4 | Hamster prion protein amino acid sequence | 2d |
| 5 | Bovine prion protein amino acid sequence | 2e |
| 6 | Elk prion protein amino acid sequence | 2f |
| 7 | YML | |
| 8 | GGYMLGS | |
| 9 | GGYMLG | |
| 10 | GYMLGS | |
| 11 | GGYML | |
| 12 | YMLGS | |
| 13 | GYML | |
| 14 | YMLG | |

The present invention will be further illustrated in the following examples. However it is to be understood that these examples are for illustrative purposes only, and should not be used to limit the scope of the present invention in any manner.

### Example 1

### Methods

General references: 263K hamster-adapted prions are described by Kimberlin et al., 1978. RML mouse-adapted prions are described by Chandler, R. L. (1961) (Lancet 1, 1378-1379). These may be used to infect mice or hamsters, using methods known in the art, for example those of Bueler H et al., 1993 (Cell 73:1339-1347), Oldstone et al., 2002, or Meade-White et al., 2009. Bolton et al., 1987 describe methods that may be used for isolation and purification of scrapie agent. Carlson et al., 1986 (Cell, 46:503-511) describes methods that may be used for clinical diagnosis of scrapie in mice and hamsters. Various transgenic mice overexpressing, partially expressing or lacking expression of PrP are described by Fischer et al., 1996 (EMBO J. 15:1255-1264) and Weissmann et al., 2003 (British Medical Bulletin 66:43-60).

Brain and Spleen Homogenate Preparation

Brain tissues (normal and scrapie-infected mouse or hamster) were processed and analyzed as modified from Fischer et al 2000 (Nature 408:479-483). Briefly, 10% homogenates were made in PBS, 0.5% deoxycholate (Sigma), 0.5% NP-40. The total protein concentration in the homogenate was determined by BCA assay (Pierce) and adjusted to 5 mg/ml with homogenization buffer. For detection of PK-resistant material, 1.5 μl of homogenate was incubated with, or without, 0.15 μg PK (Sigma) at 37° C. for 60 minutes. Digestion was halted by addition of 20 mM PMSF (Sigma).

Magnetic Bead Antibody Conjugation and Immunoprecipitation

Antibodies were conjugated to magnetic beads and used for immunoprecipitation experiments as modified from Paramithiotis, 2003. Briefly, 7×10⁸ magnetic beads (in 1 ml PBS) (Dynal; Lake Success, New York) were coupled according to the manufacturer's instructions to: 1A1, 8B4, 4E4 or IgM isotype control. Conjugated beads were washed and blocked according to the manufacturer's recommendations, then resuspended in 1 ml of PBS.

104 of antibody-coupled beads were incubated with 1 μL of 10% brain homogenate in 6% detergent (3% Tween20 and 3% NP40 in PBS) for 3 hr at room temperature. Magnet-captured immune complexes were washed 3 times with 4% detergent (2% Tween20 and 2% NP40 in PBS), boiled in 4% SDS without reducing agents, and resolved on 15% Trisglycine or 4-12% bis-tris acrylamide gels (Invitrogen).

Immunoblotting

Immunoblotting was performed as described (Paramithiotis et al., 2003). Proteins were transferred onto PVDF membranes (Invitrogen). Membranes were blocked with 5% (w/v) dried non-fat milk. All incubations were done in TBST (25 mM Tris-HCl, 0.2 M NaCl, 0.5% Tween-20). Peroxidase activity was detected by chemiluminescence; enhanced ECL (Amersham) or superWest Dura (Pierce; Rockford, Ill.).

### Example 2

### Antibody Generation

Antibodies were generated by immunizing Balb/c mice with the KLH-coupled peptide GGYMLGS (SEQ ID No. 8) which corresponds to amino acids 126-132 of the human prion protein. This peptide is located in the first beta strand and has been predicted to be unfolded and accessible in misfolded PrP^{Sc}, but not native PrP^{C}. Monoclonal antibodies were generated by standard hybridoma methods. Antibodies were selected based on binding (by ELISA) to the immunogen peptide coupled to BSA.

More particularly, a peptide with the amino acid sequence Acetyl-Cys-GGYMLGS-NH2 was synthesized, conjugated to KLH, and injected intramuscularly into rabbits using well known techniques. The N-terminal cysteine residue was added to allow conjugation of the peptide with the protein carrier. The amino group of the peptide was blocked by acetylation, and the carboxylic group of the peptide was blocked by amidation. Peptides were synthesized using solid phase peptide synthesis methods either manually or automated (MPS396 peptides synthesizer, Advanced ChemTech). Coupling of amino acid residues was accomplished using Fmoc peptide synthesis chemistry (Fields et al., 1990, IJPPR 35, 161). Syntheses were performed on Wang or on amide Rink resins, with full side chain protection of amino acids. After synthesis, the peptides were cleaved from the resin using the Reagent K as a cleavage mixture: water (2.5%), TIS (2.5%), EDT (2.5%), TFA (92.5%). The peptides were then precipitated with cold diethyl ether. The precipitates were centrifuged, washed three times with diethyl ether, dissolved in 20%-50% AcCN/water mixture, and lyophilized. Analysis of crude products was performed using analytical RP-HPLC and electrospray MS. The crude peptide was purified by Rp-HPLC (reverse phase high performance liquid chromatography) on a Vydac C18 column, 2.5×25 cm, using a linear gradient of 10-50% acetonitrile in water, with 0.06% TFA (1%/min gradient, 10 ml/min flow rate), with monitoring by UV at 215 nm and 254 nm. Peptides were coupled to a carrier, in this case Keyhole limpet hemocyanin (KLH). Other carriers useful for such coupling include, without limitation, albumin, or ovalbumin, 8map, or lysozyme. Coupling was effected via a thioether linkage to the mercapto group of the cysteine. Coupling to KLH was performed as follows. 10 mg of the peptide was dissolved in 2 ml of phosphate buffered solution (PBS 1x). 1 ml of KLH (pierce products #77100) was added to the peptide solution and stirred (1 mole of peptide/50 amino acids). The KLH concentration was 10 mg/ml. 20 ul of glutaraldehyde (25% aqueous solution) was added to the peptide/carrier solution with constant stirring, incubated for 1 hour, after which a glycine stop solution was added. The peptide/carrier conjugate was separated from the peptide by dialysis against PBS.

The generation of monoclonal antibodies was carried out as follows. Mice were immunized with baculovirus supernatant containing mouse PrP-AP fusion protein in complete Freund's adjuvant, then boosted 2 weeks later with the same antigen in incomplete Freund's adjuvant. Two weeks after that immunization the mice were boosted with a mixture of PrP-AP supernatant plus 100 ug of KLH-CGGYMLGS conjugate. Splenocytes from these mice were fused to the FO murine B cell line (ATCC CRL-1646) to generate specific hybridoma clones.

Mouse monoclonal antibodies were produced as ascites, and purified using a protein A column kit (Pierce) according to the manufacturer's instructions. Briefly, a sample of ascites was diluted with binding buffer at a 1:1 final ratio. The sample was then added to the top of the column, which had been previously equilibrated with binding buffer, and allowed to flow through the matrix. The pass-through material was collected and the column washed with 5 volumes of binding buffer. Mild elution buffer was added to the column to release the bound antibody from the matrix. All the antibodies were collected in 1 ml fractions, which were analyzed by BCA to determine total protein content and SDS-PAGE electrophoresis to establish the degree of antibody purity. The fraction containing desired antibody was desalted by passing it through a D-salt column (Pierce). The antibody fraction was allocated and stored at −80 C. in PBS.

The hybridoma producing the murine monoclonal antibody designated 1A1 was deposited under terms of the Budapest Treaty with International Depositary Authority of Canada on Feb. 26, 2010, under accession number 260210-01.

In a like manner, antibodies, and hybridomas that produce them, are raised against other YML-containing peptides including those having SEQ ID Nos. 7 and 9-14.

### Example 3

### Immunoprecipitation of PrP^{Sc}

Immunoprecipitated samples were analyzed by Western blotting (FIG. 1) with 6D11-biotin as the primary antibody (1:5000) and Strep-HRP as the secondary antibody (1:5000). 8B4-bead acted as a positive control and was able to immunoprecipitate PrP from all the brain homogenate samples except the PrP Knocked Out mouse (K/0). Beads only, IgM-isotype-beads and 4E4-beads acted as negative controls and as expected, no PrP was immunoprecipitated, except two very faint bands in the RML and 263K lanes as immunoprecipitated by the 4E4. 1A1, an IgM antibody that was raised against the beta-1 strand of PrP, was able to immunoprecipitate scrapie proteins from both RML (mouse scrapie strain) and 263K (hamster scrapie strain). There is a faint band in the Tg20 lane possibly due to a small expression of misfolded PrP in the overexpression PrP mouse brain. Our data indicated that 1A1 was able to recognize only the scrapie PrP, but not the wild type PrP in both mouse and hamster brain.

### Example 4

### YML Tumour Targets

The 1A1 antibody was tested for its ability to bind to both normal and tumor cells. As a control, the anti-PrP^{C} antibody 6D11 was used to identify the level of expression of PrP on each cell type. The ability of antibodies to bind to cells was observed by flow cytometry using HUVEC (human umbilical vein endothelial cells) as the normal cell type. Data is shown for binding to eight types of tumor cells. Five of the cancer cells tested are immortalized cell lines from mice (B16—melanoma, NSC34—motor neuron/neuroblastoma hybrid) and humans (HL60—promylocytic leukemia, MO3.13—oligodendrocyte/muscle hybrid, SiHa—cervical carcinoma). The remaining cancer cells tested are primary tumor cells that have been propagated by the Living Tumor Laboratory (LTL) at the British Columbia Cancer Agency. Primary human tumors are propagated under the kidney capsules of immunodeficient mice. This allows the original tumor architecture and phenotype to remain consistent with the originally harvested tumor. The three tumors that have been tested for binding to the 1A1 antibody are LTL-013 (large diffuse B-Cell lymphoma), LTL-257 (colorectal sarcoma) and LTL-323 (melanoma).

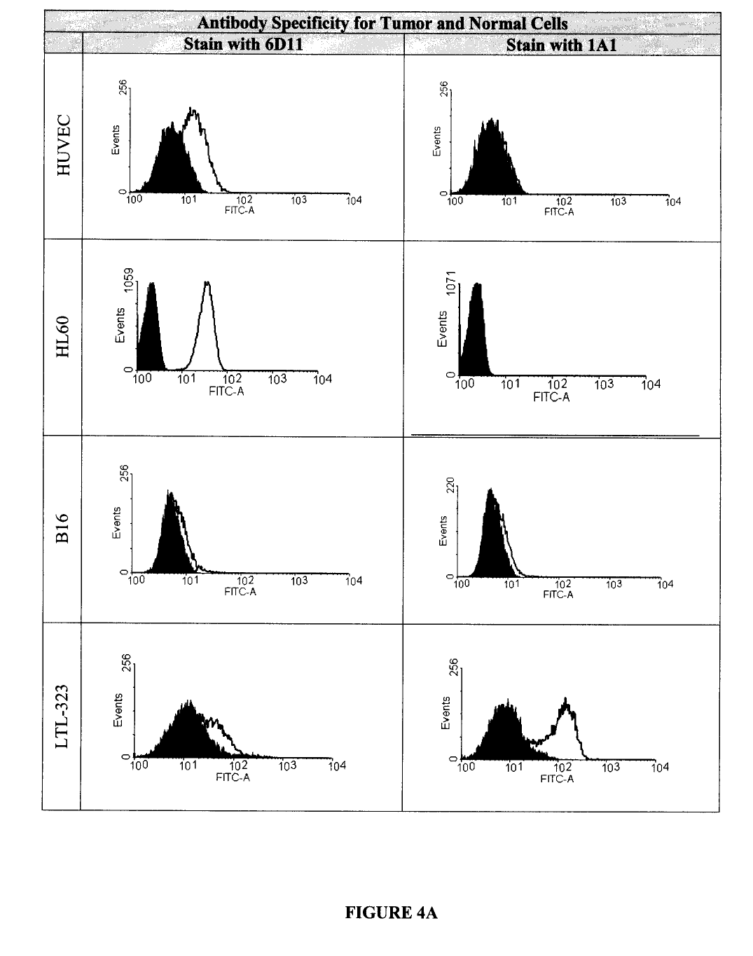

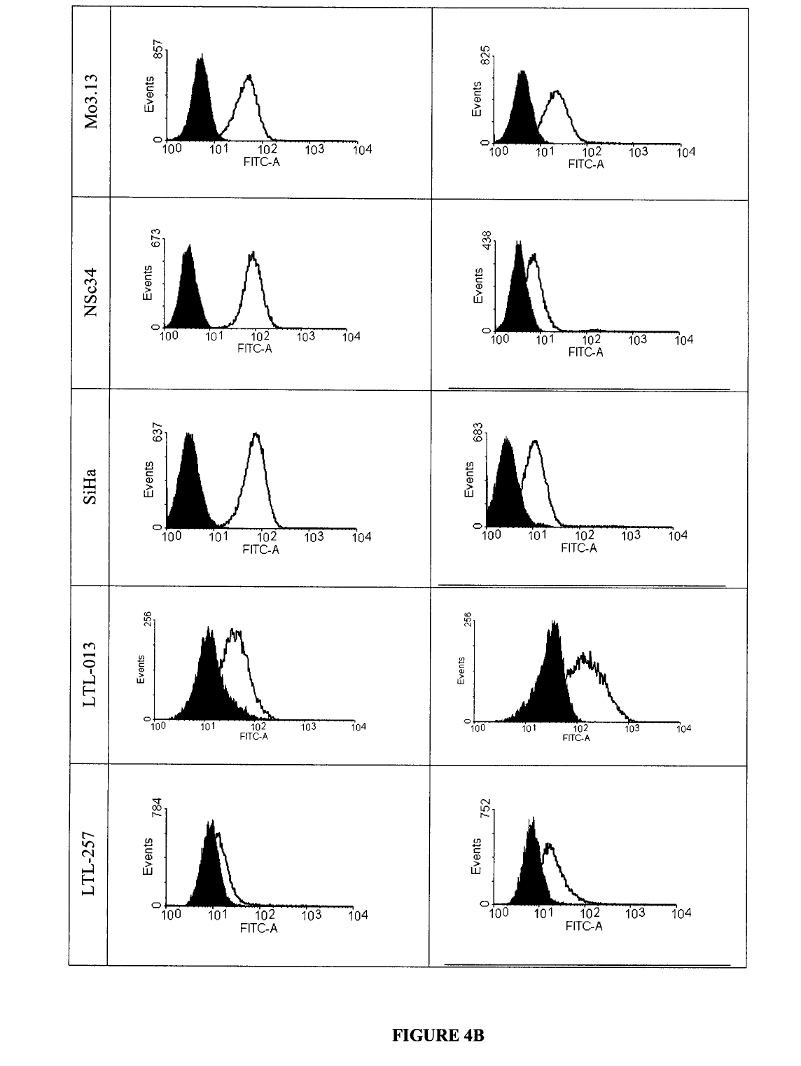

Results are shown in FIG. 4. The histogram in dark is the staining observed with the isotype control antibody and the histogram shown as a black line is the staining with the specific antibody 6D11 or 1A1 (as indicated). PrP is expressed on all nine cell types shown. The 1A1 antibody shows minimal binding both to the normal HUVEC cells and the leukemia cells (HL60). The 1A1 antibody shows detectable binding to the other seven tumour cells shown.

### Example 5

### Efficacy in a Tumor Model

The 1A1 antibody was tested for its ability to modify growth of a murine melanoma tumor (B16) in female C57Bl/6 mice. On day 0 of the study, 3×10⁵ tumour cells were implanted subcutaneously into the flank of 12 mice. The mice were randomly assigned to two treatment groups. Group 1 was treated with PBS. Group 2 was treated with 1A1 antibody at 10 mg/kg. Mice were treated on days −1, 2 and 5.

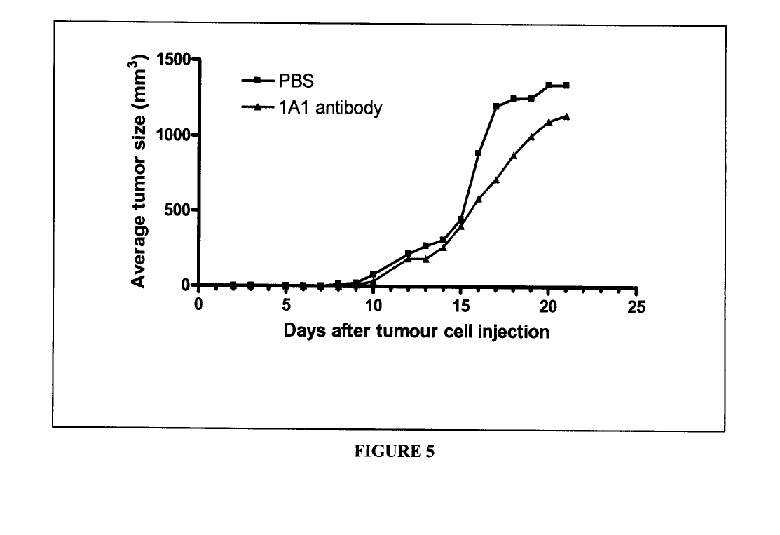

Tumour growth was monitored by measuring tumour dimensions with calipers beginning on day 2. Tumour length and width measurements were obtained and tumour volumes were calculated according to the equation L×W²/2 with the length (mm) being the longer axis of the tumour. Mice were sacrificed once tumour burden was high, according to standard animal care procedures. FIG. 5 shows the progression of tumour growth in both treatment groups in which the tumour volume prior to termination is carried through to the end. There is a significant difference in tumour growth between the two groups (paired t-test=0.012; Wilcoxin=0.007), indicating that a therapeutic effect of the 1A1 antibody has occurred.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

One or more currently preferred embodiments of the invention have been described by way of example. The invention includes all embodiments, modifications and variations substantially as hereinbefore described and with reference to the examples and figures.

It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

1. An antibody or fragment thereof that binds a YML epitope of a misfolded PrP.

2. The antibody of claim 1, wherein the antibody selectively binds a PrP^{Sc}.

3. The antibody of claim 1 or 2, wherein the antibody does not specifically bind a PrP^{C}.

4. The antibody of claim 1, wherein the epitope is present in a sequence selected from one or more of the group consisting of: GGYMLGS, GGYMLG, GYMLGS, GGYML, YMLGS, GYML and YMLG (SEQ ID NOs: 8-14).

5. The antibody of claim 1, wherein the antibody is a monoclonal antibody.

6. The antibody of claim 1, wherein the antibody is a polyclonal antibody.

7. The antibody of claim 1, wherein said antibody is an IgG, IgM, IgE, IgD, or IgA.

8. The antibody of claim 1, produced by culturing the hybridoma deposited with the International Depositary Authority of Canada under accession number 260210-01.

9. An immunoconjugate comprising an antibody according to claim 1 and, conjugated therewith, an agent selected from one or more of a group consisting of a detectable label and a cytotoxin.

10. An immunogenic peptide directed against an antibody that binds selectively to misfolded PrP, the peptide comprising a YML sequence.

11. The peptide of claim 10, wherein the peptide is selected from one or more of the group consisting of the sequence of SEQ ID NO: 7, SEQ ID NO:8, SEQ ID NO:9, SEQ ID NO:10, SEQ ID NO: 11, SEQ ID NO: 12, SEQ ID NO:13 and SEQ ID NO:14.

12. A peptide according to claim 10, further comprising an immunogenic carrier to enhance immunogenicity of said peptide.

13. A composition comprising the antibody of claim 1.

14. A composition comprising the immunoconjugate of claim 9.

15. A composition comprising a peptide according to claim 10.

16. (canceled)

17. (canceled)

18. (canceled)

19. (canceled)

20. (canceled)

21. (canceled)

22. (canceled)

23. (canceled)

24. A method of treating or preventing a disease or disorder associated with misfolded PrP, the method comprising administering a therapeutically effective amount of an antibody according to claim 1, an immunoconjugate according to claim 9, or a peptide according to claim 10, to a subject in need thereof.

25. A method of immunizing a subject with, or at risk for, a disease or disorder associated with misfolded PrP, the method comprising administering a therapeutically effective amount of a vaccine comprising a peptide according to claim 10 to a subject in need thereof.

26. The method of claim 24, wherein the disease or disorder is associated with PrP^{Sc}.

27. The method according to claim 24, wherein the disease or disorder is selected from the group consisting of Gerstmann-Sträussler-Scheinker disease (GSS), familial Creutzfeldt-Jakob disease, sporadic Creutzfeldt-Jakob disease, iatrogenic Creutzfeldt-Jakob disease, variant Creutzfeldt-Jakob disease, fatal familial insomnia, scrapie, Kuru, spongiform encephalopathy, transmissible mink encephalopathy, chronic wasting disease, feline spongiform encephalopathy, and exotic ungulate encephalopathy.

28. The method according to claim 24, for the treatment of a tumour comprising a tumorigenic cell expressing a misfolded PrP.

29. The method according to claim 28, wherein the tumour has a YML+ phenotype.

30. A hybridoma cell line that produces a monoclonal antibody that binds to a YML epitope of a misfolded PrP.

31. The hybridoma cell line of claim 30, wherein the misfolded PrP is a PrP^{Sc}.

32. A hybridoma cell line according to claim 30, which is the hybridoma deposited with the International Depositary Authority of Canada under accession number 260210-01, and progeny and derivatives thereof.

33. The hybridoma cell line of claim 30, wherein said YML epitope is present in sequence GGYMLGS (SEQ ID NO: 8).

34. A method for detecting a misfolded PrP in a biological sample, comprising:
(a) contacting a biological sample with the antibody of claim 1, or the immunoconjugate of claim 9, under conditions that allow for the formation of a complex between said antibody or said immunoconjugate and said misfolded PrP, and
(b) detecting the complex as an indication that misfolded PrP is present in the biological sample.

35. (canceled)

36. The method of claim 34, wherein the misfolded PrP is a PrP^{Sc}.

37. A method of producing an antibody that binds a YML epitope of a misfolded PrP, the method comprising:
(a) culturing a hybridoma cell line of claim 30 under conditions that release the antibody into the culture supernatant; and
(b) isolating the antibody from the supernatant.

38. (canceled)

39. A method of producing an antibody that binds a YML epitope of a misfolded PrP, the method comprising:
(a) immunizing a subject with the peptide of claim 10; and
(b) isolating the antibody from a tissue of the subject, or from a hybridoma prepared from the tissue.

40. A kit for detecting the presence of misfolded PrP in a biological sample comprising:
(a) one or more antibodies or antisera that specifically bind the YML epitope of misfolded PrP; and
(b) instructions for its use.

41. The kit of claim 40, further comprising one or more detection reagents.


SEQUENCE LISTING (14 sequences)

SEQ ID NO: 1 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 3 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 4 - protein, 254 aa, Mesocricetus auratas
MANLSYWLLALFVATWTDVGLCKKRPKPGGWNTGGSRYPGQGSPGGNRYPPQGGGTWGQPHGGGWGQPHGGGWGQPHGGGWGQPHGGGWGQGGGTHNQWNKPNKPKTSMKHMAGAAAAGAVVGGLGGYMLGSAMSRPMLHFGNDWEDRYYRENMNRYPNQVYYRPVDQYNNQNNFVHDCVNITIKQHTVTTTTKGENFTETDVKMMERVVEQMCVTQYQKESQAYYDGRRSSAVLFSSPPVILLISFLIFLIVG

SEQ ID NO: 5 - protein, 264 aa, Bos taurus
  misc_feature (59)..(59): Xaa can be any naturally occurring amino acid
MVKSHIGSWILVLFVAMWSDVGLCKKRPKPGGGWNTGGSRYPGQGSPGGNRYPPQGGGXWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 6 - protein, 256 aa, Cervus elaphus elaphus
MVKSHIGSWILVLFVAMWSDVGLCKKRPKPGGGWNTGGSRYPGQGSPGGNRYPPQGGGSWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 7 - protein, 3 aa, Artificial Sequence
  YML epitope of misfolded human prion protein (PrP)
YML

SEQ ID NO: 8 - protein, 7 aa, Artificial Sequence
  Peptide including the YML epitope of misfolded human prion protein (PrP)
GGYMLGS

SEQ ID NO: 9 - protein, 6 aa, Artificial Sequence
  Peptide including the YML epitope of misfolded human prion protein (PrP)
GGYMLG

SEQ ID NO: 10 - protein, 6 aa, Artificial Sequence
  Peptide including the YML epitope of misfolded human prion protein (PrP)
GYMLGS

SEQ ID NO: 11 - protein, 5 aa, Artificial Sequence
  Peptide including the YML epitope of misfolded human prion protein (PrP)
GGYML

SEQ ID NO: 12 - protein, 5 aa, Artificial Sequence
  Peptide including the YML epitope of misfolded human prion protein (PrP)
YMLGS

SEQ ID NO: 13 - protein, 4 aa, Artificial Sequence
  Peptide including the YML epitope of misfolded human prion protein (PrP)
GYML

SEQ ID NO: 14 - protein, 4 aa, Artificial Sequence
  Peptide including the YML epitope of misfolded human prion protein (PrP)
YMLG